(19) United States
(12) Patent Application Publication
(10) Pub. No.: US 2024/0350527 A1
(43) Pub. Date: Oct. 24, 2024

(54) COMPOSITIONS AND METHODS FOR TREATING POST-COVID CONDITIONS OF FATIGUE

(71) Applicant: AIM ImmunoTech Inc., Ocala, FL (US)

(72) Inventors: Thomas K. EQUELS, Ocala, FL (US); David R. STRAYER, Bryn Mawr, PA (US)

(73) Assignee: AIM ImmunoTech Inc., Ocala, FL (US)

(21) Appl. No.: 18/685,067

(22) PCT Filed: Aug. 22, 2022
(86) PCT No.: PCT/US2022/075299

§ 371 Date: Feb. 20, 2024

Related U.S. Application Data
(60) Provisional application No. 63/342,562, filed on May 16, 2022.
(60) Provisional application No. 63/235,388, filed on Aug. 20, 2021.

(51) Int. Cl. A61K 31/713; A61P 43/00; C12N 15/117

(52) U.S. Cl. CPC A61K 31/713; A61P 43/00; C12N 15/117

(57) ABSTRACT

Disclosed is a method for treating a subject that has previously been infected with SARS-COV-2 and exhibiting at least one Post COVID-19 Conditions of fatigue (PCC of fatigue) symptom. The method comprises administering to the subject a therapeutically effective amount of a composition comprising therapeutic double-stranded RNA (tdsRNA). Compositions, medicaments and delivery systems comprising tdsRNA for the treatment of PCC of fatigue are also disclosed.

### BACKGROUND

While most people with COVID-19 recover within weeks of illness, some experience post-COVID symptoms long after clearance (resolution) of their COVID-19 (SARS-COV-2) infections. Even asymptomatic but SARS-COV-2 infected people may develop post-COVID conditions (referred to herein as “PCC”) in the days or weeks after SARS-COV-2 infection.

This disclosure relates to Post-COVID Conditions of fatigue (referred to herein as “PCC of fatigue”), described in more detail in the Detailed Disclosure, that is a subset of PCC symptoms. PCC of fatigue symptoms are significant because they seriously affect quality of life. A large-scale data analysis of 81,337 participants who completed an intelligence test between January and December 2020—including 12,689 individuals who had experienced COVID-19—found that people who had recovered from COVID-19 tended to experience significant reductions in their cognitive abilities. The perceived drop in IQ tended to increase with the severity of a participant's COVID-19 symptoms. According to the study's authors: “When examining the entire population, the deficits were most pronounced for paradigms that tapped cognitive functions such as reasoning, problem-solving, spatial planning and target detection whilst sparing tests of simpler functions such as working-memory span as well as emotional processing.”

PCC of fatigue in its most severe form is disabling and requires constant attention from caregivers with a devastating impact on the subject and on society. At this time, the care available to PCC of fatigue sufferers is limited to supportive care in the uncertain hope of eventual spontaneous recovery.

### SUMMARY

One embodiment is directed to a method for treating a subject that has previously been infected with SARS-COV-2 and exhibiting at least one Post COVID-19 Conditions of fatigue (PCC of fatigue) symptom comprising the steps of: administering to the subject a therapeutically effective amount of a composition comprising therapeutic double-stranded RNA (tdsRNA). In the method the tdsRNA is at least one selected from the group consisting of rIₙ·r(CₓU)ₙ (formula A); and rugged dsRNA (formula B); wherein x is at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35. In one embodiment, the subject has previously been infected with SARS-COV-2 but the infection is resolved before the administration step. That is, SARS-COV-2 is no longer detectable in the subject, or no longer detectable in the blood (e.g., peripheral blood) by nucleic acid (e.g., PCT, RT-PCR), antibody and protein-based detection methods.

In the method, there are two additional optional steps that can be performed in any order. Optional step (a) is a determining step before the administering step; the determining step may comprise determining that the subject was previously infected with SARS-COV-2 and performing the administering step if the subject was previously infected with SARS-COV-2. Determining may be, for example, detecting antibodies or other indicia of a previous SARS-COV-2 infection. Another determination method may be, for example, reviewing medical records. Optional step (b) is a determining step before the administering steps in a subject to determine if the subject has no history of chronic fatigue or chronic fatigue symptoms before being infected by SARS-COV-2 and performing the administering step if the subject has no history of chronic fatigue or chronic fatigue symptoms. This step may be performed by reviewing the subject's medical history and this step can further ensure that the subject's symptoms are PCC of fatigue symptoms. As stated above, steps (a) and step (b) may be performed in any order. Alternatively, only one of the steps may be performed. Since both steps are optional, in some embodiments, neither optional step is performed.

In any embodiment of this disclosure (e.g., methods, compositions, medicaments, medical devices, delivery systems, and the like) the subject may exhibit at least one, at least two, at least three or at least four PCC of fatigue symptom selected from the group consisting of: (1) difficulty concentrating or focusing, (2) inability to exercise or be active, (3) mental fatigue, and (4) post exertional malaise (tiredness the day after exercise). Further, in any embodiment of this disclosure (e.g., the methods, compositions, medicaments, medical devices, delivery systems), the embodiment may reduce at least one, at least two, at least three or at least four of the disclosed PCC of fatigue symptoms.

In any embodiment, the subject may be a mammal such as any mammal discussed in this disclosure. In a preferred embodiment, the subject may be a host of SARS-COV-2. For example, a host of SARS-COV-2 may be a human ACE2 receptor expressing animal. Examples of these animals may be mouse; hamster such as Mesocricetus auratus or Cricetulus griseus, or monkey such as Macaca mulatta or Macaca fascicularis. In another preferred embodiment, the subject may be a human.

In any embodiment, at least 90 wt. % of the tdsRNA may be larger than a size selected from the group consisting of: 40 basepairs; 50 basepairs; 60 basepairs; 70 basepairs; 80 basepairs; and 380 basepairs.

In any embodiment, at least 90 wt. % of the tdsRNA may be smaller than a size selected from the group consisting of: 50,000 basepairs; 10,000 basepairs; 9000 basepairs; 8000 basepairs; 7000 basepairs; and 450 basepairs.

In any formula of this disclosure and in any embodiment of this disclosure, n may be a number with a value selected from the group consisting of: 40 to 50,000; 40 to 40,000; 50 to 10,000; 60 to 9000; 70 to 8000; 80 to 7000; and 380 to 450.

For the tdsRNA of this disclosure, (1) n may be from 40 to 40,000; (2) the tdsRNA may be about 4 to about 4000 helical turns of duplexed RNA strands; and/or (3) the tdsRNA may have a molecule weight selected from the group consisting of: 2 kDa to 30,000 kDa; 25 kDa to 2500 kDa; and 250 kDa to 320 kDa.

In any embodiment, the tdsRNA may comprise rIₙ·r(C₁₁₋₁₄U)ₙ; and rugged dsRNA.

In any embodiment, the tdsRNA may be in a composition and the composition may comprise at least one pharmaceutically acceptable carrier.

In any embodiment, the administration step may be performed after the primary COVID-19 infection has been resolved-after SARS-COV-2 infection or SARS-COV-2 is no longer detectable in a subject. Detection may be analysis of blood by nucleic acid, protein, or antibody methods.

In any embodiment, administering may be performed after the subject was infected with SARS-COV-2 initially for a time before the administering step, wherein said time is selected from the group consisting of 30 days; 50 days; 2 months; 3 months; 4 months; 5 months; 6 months; 1 year; 2 years; and more than 2 years. Furthermore, the administration may be performed after the SARS-COV-2 infection is resolved.

In any method, administering or administration may be any administration method of this disclosure. In a preferred embodiment, the administration method may be at least one administering method selected from the group consisting of: nasal administration; systemic administration; and intravenous administration. Other administration methods may be intradermal administration; subcutaneous administration; intramuscular administration; intranasal administration (e.g., pulmonary airway administration); intranasal administration and oral administration; intraperitoneal administration; intracranial administration; intravesical administration; oral administration (e.g., through the mouth, by breathing through the mouth); topical administration; inhalation administration; aerosol administration; intra-airway administration; tracheal administration; bronchial administration; instillation; bronchoscopic instillation; intratracheal administration; mucosal administration; dry powder administration; spray administration; intratracheal deposition administration; intrabronchial deposition administration; bronchoscopic deposition administration; lung administration; nasal passage administration; respirable solid administration; respirable liquid administration; dry powder inhalants administration.

In any embodiment, administering may be performed by a delivery system or medical device comprising the tdsRNA. The delivery system or medical device may be any described in this disclosure and include at least one selected from the group consisting of: a nebulizer; a sprayer; a nasal pump; a squeeze bottle; a nasal spray; a syringe sprayer or plunger sprayer (e.g., a syringe providing pressure to an attached sprayer or nozzle); a swab; a pipette; a nasal irrigation device; and a nasal rinse.

In any embodiment, the tdsRNA is administered at a dosage of about 25 mg to 700 mg of tdsRNA per day; 20 mg to 200 mg of tdsRNA per day; 50 mg to 150 mg of tdsRNA per day; or 80 mg to 140 mg of tdsRNA per day.

In any embodiment, the tdsRNA may be administered at a frequency selected from the group consisting of: one dose per day, one dose every 2 days, one dose every 3 days, one dose every 4 days, one dose every 5 days, one dose a week, two doses a week, three doses a week, one dose every two weeks, one dose every 3 weeks, one dose every 4 weeks, and one dose a month. In a preferred embodiment, the tdsRNA may be administered two times a week and at 400 mg per administration. In another preferred embodiment, the tdsRNA may be administered two times a week at 200 mg per administration for the first 2 weeks, two times a week at 400 mg per administration after the first 2 weeks.

In any embodiment of this disclosure (e.g., methods, compositions, medicaments, medical devices, delivery systems, and the like) the embodiment may reduce at least one, at least two, at least three, or at least 4 symptoms selected from the group consisting of: difficulty concentrating or focusing, inability to exercise or be active, mental fatigue, and post exertional malaise (tiredness the day after exercise),

Another embodiment is directed to a composition for treating PCC of fatigue or relieving a symptom thereof in a subject comprising at least one selected from the group consisting of rIₙ·r(CₓU)ₙ (formula A); rugged dsRNA (formula B); wherein x is at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35. For example, the tdsRNA may be is at least one selected from the group consisting of rIₙ·r(C₁₁₋₁₄U)ₙ; and rugged dsRNA. The composition may be a medicament.

Another embodiment is directed to the use of the composition of this disclosure in the manufacture of a medicament for treating PCC of fatigue or relieving at least one, at least two, at least three, or at least four symptoms thereof. The medicament made by this process is also an embodiment of this disclosure.

Another embodiment is directed to a composition of this disclosure for treating PCC of fatigue or relieving at least one, at least two, at least three, or at least four symptoms thereof.

Another embodiment is directed to a delivery system comprising the tdsRNA composition described above or anywhere in this disclosure.

In the method or any embodiment, the subject may have no history of chronic fatigue before SARS-COV-2 infection; the subject may be seropositive for an anti-SARS-COV-2 antibody; the subject may be seronegative for the anti-SARS-COV-2 antibody; the subject may be not infected with SARS-COV-2 during the administering step; the subject may be infected with SARS-COV-2 during the administering step; or the subject may be not hospitalized for SARS-COV-2 infection during the administering step.

In the method or any embodiment, the subject (1) may not have PCC of fatigue symptoms before being infected with SARS-COV-2; or (2) the subject may not have PCC of fatigue symptoms for at least 1 year; 2 years; 3 years; 4 years; or 5 years before being infected with SARS-COV-2; or (3) the subject may develop the PCC of fatigue symptoms during or after being infected with SARS-COV-2.

In the method or any embodiment, the at least one PCC of fatigue symptom may have lasted from infection to the administering step, or the at least one PCC of fatigue symptoms have lasted for a time selected from the group consisting of: at least 1 month; at least 2 months; at least 3 months; at least 4 months; at least 5 months; at least 6 months; at least 1 year; at least 2 years; and more than 2 years.

In any embodiment, the rugged dsRNA may comprise a single strand comprised of r(C₄₋₂₉U)ₙ, r(C₁₁₋₁₄U)ₙ, or r(C₁₂U)ₙ; and an opposite strand comprised of r(I)ₙ; wherein the single strand and the opposite strand do not base pair the position of the uracil base, and wherein the single strand and the opposite strand are partially hybridized.

In any embodiment, the rugged dsRNA may have a molecular weight of about 250 kDa to 500 kDa; or each strand of the rugged dsRNA is from about 400 to 800 basepairs in length; or the rugged tdsRNA has about 30 to 100 or 30-60 helical turns of duplexed RNA.

In any embodiment, the rugged dsRNA may be resistant to denaturation under conditions that are able to separate hybridized poly (riboinosinic acid) and poly (ribocytosinic acid) strands (rIₙ·rCₙ).

In any embodiment, the rugged dsRNA may be an isolated double-stranded ribonucleic acid (dsRNA) enzymatically active under thermal stress comprising: each strand with a molecular weight of about 250 kDa to about 500 kDa, 400-800 basepairs, or 30 to 60 helical turns of duplex RNA; a single strand comprised of poly (ribocytosinic₄₋₂₉ uracilic acid) and an opposite strand comprised of poly (riboinosinic acid); wherein the two strands do not base pair the position of the uracil base; wherein the two strands base pair the position of the cytosine base; and wherein said strands are partially hybridized.

In any embodiment, the tdsRNA may (1) comprises 0.1-12 mol % rugged dsRNA; (2) comprise 0.1-5 mol % rugged dsRNA.

In any embodiment, the tdsRNA may be a stabilized tdsRNA. The stabilized tdsRNA may be dried tdsRNA, preferably lyophilized tdsRNA. The tdsRNA may be complexed with a stabilizing polymer. The stabilizing polymer is at least one selected from the group consisting of: polylysine; polylysine and carboxymethylcellulose; polyarginine; polyarginine and carboxymethylcellulose.

In any embodiment that comprises a delivery system, the delivery system may be selected from the group consisting of a nebulizer; a sprayer; a nasal pump; a squeeze bottle; a nasal spray; a syringe sprayer or plunger sprayer (a syringe providing pressure to an attached sprayer or nozzle); a swab; a pipette; a nasal irrigation device; a nasal rinse; a peripheral venous catheter; a needle, a tube, a line, a central venous catheter, a peripherally inserted central catheter, a tunneled catheter, or an implanted port.

Each of the examples, and each part of the examples, in the Example section, is also an embodiment of this disclosure.

### DETAILED DESCRIPTION

### 1. PCC of Fatigue

PCC of fatigue is a subset of post-COVID symptoms that is present even when a SARS-COV-2 infection is resolved. The severity or presence of PCC of fatigue is not necessarily correlated to the severity or duration of the initial SARS-COV-2 infection. For example, a younger patient with only mild congestion during their infection can go on to develop severe PCC of fatigue. In these cases, the PCC of fatigue symptoms can feel more severe and debilitating than the initial infection. PCC of fatigue patients are also (or can be) negative when tested for the presence of SARS-COV-2 (i.e., the SARS-COV-2 infection has resolved) showing that PCC of fatigue symptoms are not a result of continued SARS-COV-2 infection.

While PCC of fatigue may be less severe than an acute SARS-COV-2 infection and may not require hospitalization, it may be debilitating enough that a subject suffering from PCC of fatigue may not be able to work or work as productively as before the onset of PCC of fatigue. Subjects with PCC of fatigue may be unable to live an active lifestyle, unable to work, and even unable to perform everyday tasks necessary for survival. Sometimes the patients develop PCC of fatigue after a period of time (e.g., 4 to 8 weeks, 6 months, 9-12 months) after the resolution of their infection.

PCC of fatigue symptoms can include, for example, one, at least one, two, three or four of the following: (1) difficulty concentrating or focusing, (2) inability to exercise or be active, (3) mental fatigue, and (4) post exertional malaise (tiredness the day after exercise). In a preferred embodiment, the PCC of fatigue symptoms may be: inability to exercise or be active, mental fatigue, and post exertional malaise (tiredness the day after exercise).

This disclosure refers to a surprising discovery that the symptoms of PCC of fatigue can be treated (e.g., reduced) rapidly, within one week or two weeks, after administration with tdsRNA. Where the administration was made intravenously, these treatments were effective in reducing or eliminating one or more PCC of fatigue symptoms within one week, two weeks, 4 weeks, 8 weeks, 12 weeks or 24 weeks and the symptoms remain reduced after treatment or as long as the treatment is continued.

### 2. Definitions

“COVID-19” or “COVID19” is a disease caused by an infection of a subject by the “SARS-COV-2” virus.

This disclosure relates to, inter alia, tdsRNA. tdsRNA can also be called “therapeutic dsRNA,” or “therapeutic double-stranded RNA” and these terms have the same meaning. In this section, or anywhere in this disclosure, a reference to tdsRNA would include, at least, a reference to a composition comprising tdsRNA or a medicament comprising tdsRNA. Further, any reference to tdsRNA would include at least rintatolimod (also called AMPLIGEN®). Rintatolimod and AMPLIGEN® have the same meaning and can be used interchangeably.

“r” and “ribo” have the same meaning and refer to ribonucleic acid or the nucleotides or nucleosides that are the building block of ribonucleic acid.

RNA consists of a chain of linked units called nucleotides. This disclosure relates mostly to RNA and, therefore, unless otherwise specified, the nucleotides and bases expressed refers to the ribo form of the nucleotide or base (i.e., ribonucleotide with one or more phosphate groups). tdsRNA does not contain DNA. Therefore “A” refers to rA or adenine, “U” refers to rU or uracil, “C” refers to rC or cytosine, “G” refers to rG or guanine, “I” refers to rl or inosine, “rN” refers to rA, rU, rC, rG or rI. Each of these (i.e., A, U, C, G, I) may have one or more phosphate groups as discussed above depending on whether they are part of a chain (i.e., RNA) or free (nucleoside, nucleotide, etc.).

“n” is a positive number and refers to the length of ssRNA or dsRNA or to the average length of a population of ssRNA or dsRNA. “n” can be a positive integer when referring to one nucleic acid molecule or it can be any positive number when it is an average length of a population of nucleic acid molecules.

An RNA may have a ratio of nucleotides or bases. For example, r(C₁₂U)ₙdenotes a single RNA strand that has, on average 12 C bases or ribonucleotides for every U base or ribonucleotide. As another example, r(C₁₁₋₁₄U)ₙ denotes a single RNA strand that has, on average 11 to 14 C bases or ribonucleotides for every U base or ribonucleotide.

Formulas: As an example, the formula “rIₙ·r(C₁₂U)ₙ” can be expressed as riboIₙ·ribo(C₁₂U)ₙ, rIₙ·ribo(C₁₂U)ₙ, or riboIₙ·r(C₁₂U)ₙ, refers to a double-stranded RNA with two strands. One strand (rIn) is poly ribo-inosine of n bases in length. The other strand is ssRNA of random sequence of C and U bases, the random sequence ssRNA is n bases in length, and a ratio of C bases to U bases in the random sequence ssRNA is about 12 (i.e., mean 12 C to 1 U). The terms “r” and “ribo” have the same meaning in the formulas of the disclosure. Thus, rI, riboI, r (I) and ribo (I) refer to the same chemical which is the ribose form of inosine. Similarly, rC, riboC, r (C) and ribo (C) all refer to cytidine in the ribose form which is a building block of RNA. rU, riboU, r (U) and ribo (U) all refer to Uracil in the ribose form which is a building block of RNA.

The “·” symbol indicates that one strand of the dsRNA is hybridized (hydrogen-bonded) to the second strand of the same dsRNA. Therefore, rIₙ·r(C₁₂U)ₙis double-stranded RNA comprising two ssRNA. One ssRNA is poly (I) and the other ssRNA is poly (C₁₂U). It should be noted that while we referred to the two strands being hybridized, not 100% of the bases form base pairing as there are some bases that are mismatched. Also, because rU does not form base pairing with rI as well as rC form base paring with rI, rU provides a focus of hydrodynamic instability in rIₙ·r(C₁₂U)ₙ at the locations of the U bases.

As another example, the formula “rIₙ·r(C₁₁₋₁₄U)ₙ” refers to the same dsRNA except that a ratio of C bases to U bases one strand is about 11 to about 14. That is, the ratio can be 11, 12, 13 or 14 or any value between 11 and 14. For example, when half of the strands are r(C₁₂U)ₙ and half of the strands are r(C₁₃U)ₙ, the formula would be r(C_{12.5}U)ₙ.

The dsRNA (tdsRNA) and ssRNA of this disclosure are homopolymers (e.g., a single-stranded RNA where every base is the same) or heteropolymers (e.g., a single-stranded RNA where the bases can be different) of limited base composition. The tdsRNAs are not mRNA and are distinct from mRNA in structure. For example, the ssRNA and dsRNA are preferably missing one or all of the following: (1) 5′ cap addition, (2) polyadenylation, (3) start codon, (4) stop codon, heterogeneous protein-coding sequences, and (5) spice signals.

As used herein, the term “substantially free” is used operationally in the context of analytical testing of the material. Preferably, purified material is substantially free of one or more impurities. In a preferred embodiment, the tdsRNA of this disclosure is substantially free (e.g., more than 0% to less than 0.1%) or completely free (0%) of dI/dI dsRNA or dCdU/dCdU dsRNA. In other words, the tdsRNA is substantially free or completely free (0%) of homodimers of polymer 1 or homodimers of polymer 2. Substantially free in this context would be considered to be more than 0% but less than 1%, less than 0.5%, less than 0.2%, less than 0.1%, or less than 0.01% of a contaminant such as (1) dI/dI (polymer 1/polymer 1) dsRNA, dCdU/dCdU (polymer 2/polymer 2) dsRNA.

The terms “intranasal administration” or “intranasally,” as used herein, refer to a route of delivery of an active compound to a subject by spraying into the nose of the subject.

Intravenous (i.v. or IV) administration refers to the administration directly to the vein of a subject with a needle, a tube, a line, a central venous catheter, a peripherally inserted central catheter, tunneled catheter, or an implanted port. IV may be performed by IV push or by infusion. Infusion may be, for example, by pump infusion or drip infusion.

A particle, droplet, or an aerosol, and the like as delivered in this disclosure may be a liquid suspension particle or a dry particle.

Active ingredients or active agents are used interchangeably and include any active ingredient or active agent described in this disclosure including, at least, tdsRNA.

The double-stranded RNAs described in this disclosure are therapeutic double-stranded RNA, abbreviated as “tdsRNA.” tdsRNA includes, at least, rintatolimod (AMPLIGEN®) which is a tdsRNA of the formula rIₙ·r(C₁₂U)ₙ). tdsRNA may be stored or administered in a pharmaceutically acceptable solution such as Water For Injection (WFI) or Phosphate Buffered Saline (PBS).

The tdsRNA may be a tdsRNA produced by any of the methods of this disclosure—referred to herein as the “tdsRNA Product” or “tdsRNA”—the two terms have the same meaning. tdsRNA can be represented by one or more of the formulas below in any combination:

rIₙ·r(CₓU)ₙ (formula A)

rugged dsRNA (formula B)

For example, the tdsRNA may be formula A only, formula B only, or formula A and formula B only.

In any embodiment, x may be at least one selected from the group consisting of: 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29 (4 to 29), 4-30 (4 to 30), 4-35 (4 to 35), 11-14 (11 to 14), 30-35 (30 to 35). Of these, x=12, and x=11-14 (x may be any value between 11 to 14) are preferred.

In these formulas A to B, and in other formulas, where there is no subscript next to a base, the default value is “1.” For example, in the formula rIₙ·r(C₁₂U)ₙ, there is no subscript following “U,” it is understood that rIₙ·r(C₁₂U)ₙ is the same as rIₙ·r(C₁₂U₁)ₙ, and the formula is meant to convey that for the strand denoted as r(C₁₂U₁)ₙ, there are twelve rC base for every rU base. Thus, x is also a ratio of the bases of one strand of the tdsRNA. The length of the tdsRNA strand is denoted as a lowercase “n” (e.g., rIₙ·r(C₁₂U)ₙ). The subscript n is also the length of each individual single-stranded nucleic acid. Since tdsRNA is double-stranded, n is also the length of the double-stranded nucleic acid—i.e., the length of the tdsRNA. For example, rIₙ·r(C₁₂U)ₙ indicates, inter alia, a double-stranded RNA with each strand with a length of n.

In another aspect, the tdsRNA may be a rugged dsRNA (formula B).

In one embodiment, tdsRNA is at least one selected from the group consisting of formula A, and formula B. In another embodiment, tdsRNA comprises formula A only. In one preferred embodiment, tdsRNA comprises formula B only. In another embodiment, tdsRNA comprises formula A and formula B (rugged dsRNA) only.

In another aspect, at least 70%, at least 80%, or at least 90% of the tdsRNA may have a molecular weight of between 400,000 Daltons to 2,500,000 Daltons. Where the term percent (“%”) is used, the percent may be weight percent or molar percent.

In another aspect, the tdsRNA comprises a first ssRNA and a second ssRNA and each of these first ssRNA or second ssRNA may contain one or more strand breaks.

In another aspect, the tdsRNA has the property that greater than about 90%, greater than 95%, greater than 98%, greater than 99%, or 100% of the bases of the RNA are in a double-stranded configuration.

In any aspect, the tdsRNA may be in a therapeutic composition comprising, for example, a tdsRNA, and a pharmaceutically acceptable excipient (carrier).

One embodiment of tdsRNA is directed to rintatolimod (AMPLIGEN®), which is a tdsRNA of the formula rIₙ·r(C₁₂U)ₙ.

In a preferred embodiment, the tdsRNA are of the general formula rIₙ·r(C₁₁₋₁₄, U)ₙ and are described in U.S. Pat. Nos. 4,024,222 and 4, 130,641 (which are incorporated by reference herein) or synthesized according to this disclosure.

tdsRNA (e.g., rintatolimod) has undergone extensive clinical and preclinical testing. It has been well-tolerated in clinical trials enrolling over 1,200 patients with over 100,000 doses administered and there have been no rintatolimod-related deaths. Two placebo-controlled, randomized studies show no increase in serious adverse events compared to placebo. Favorable safety profiles have been seen for intraperitoneal, intravenous, and intranasal routes of administration of tdsRNA.

### 3.1 Length of tdsRNA

The length of the tdsRNA, may be represented by bases for one strand of the tdsRNA or in basepairs for both strands of the tdsRNA. It is understood that in some embodiments that not all of the bases (e.g., U and I) are in basepaired configuration. For example, rU bases do not pair as well as rC bases to inosine.

The length of the tdsRNA may be measured by (1) bases or basepairs, (2) molecular weight which is the weight of the double-stranded tdsRNA (e.g., Daltons) or (3) turns of the double-stranded RNA. These measurements can be easily interconverted. For example, it is generally accepted that there are about 629 Daltons per base pair.

“n” represents length in units of basepair or basepairs (abbreviated as bp regardless of whether it is singular or plural) for double-stranded nucleic acid. “n” can also represent bases for single-stranded RNA. Because “bp” represents singular or plural, it is the same as “bps” which is another representation of basepairs.

The tdsRNA can have the following values for its length “n” (in bases for single strand or basepairs for double strands): 4-5000, 10-50, 10-500, 10-40,000, 40-40,000, 40-50,000, 40-500, 50-500, 100-500, 380-450, 400-430, 400-800 or a combination thereof. Expressed in molecular weight, the tdsRNA may have the following values: 30 kDa to 300 kDa, 250 kDa to 320 kDa, 270 kDa to 300 kDa or a combination thereof. Expressed in helical turns, the tdsRNA may have 4.7 to 46.7 helical turns of duplexed RNA, 30 to 38 helical turns of duplexed RNA, 32 to 36 helical turns of duplexed RNA or a combination thereof.

The length may be an average basepair, average molecular weight, or an average helical turns of duplexed RNA and can take on integer or fractional values.

### 3.2 Rugged dsRNA (a form of tdsRNA)

Rugged dsRNA is a tdsRNA that is resistant to denaturation under conditions that are able to separate hybridized poly (riboinosinic acid) and poly (ribocytosinic acid) strands (that is, rIₙ·rCₙ strands). See, U.S. Pat. Nos. 8,722,874 and 9,315,538 (incorporated by reference) for a further description of Rugged dsRNA and exemplary methods of preparing such molecules.

In one aspect, a rugged dsRNA can be an isolated double-stranded ribonucleic acid (dsRNA) which is resistant to denaturation under conditions that are able to separate hybridized poly(riboinosinic acid) and poly(ribocytosinic acid) strands, wherein only a single strand of said isolated dsRNA comprises one or more uracil or guanine bases that are not base-paired to an opposite strand and wherein said single strand is comprised of poly(ribocytosinic₃₀₋₃₅uracilic acid). Further, the single strand may be partially hybridized to an opposite strand comprised of poly(riboinosinic acid). In another aspect, rugged dsRNA may be an isolated double-stranded ribonucleic acid (dsRNA) which is resistant to denaturation under conditions that are able to separate hybridized poly(riboinosinic acid) and poly(ribocytosinic acid) strands.

In another aspect, Rugged dsRNA, has at least one of the following: r(Iₙ)·r(C₄₋₂₉U)ₙ, r(Iₙ)·r(C₁₂U)ₙ, r(Iₙ)·r(C₁₁₋₁₄U)ₙ, r(Iₙ)·r(C₃₀U)ₙ, or r(Iₙ)·r(C₃₀₋₃₅U)ₙ. In another aspect, Rugged dsRNA may have a size of 4 bps to 5000 bps, 40 bps to 500 bps, 50 bps to 500 bps, 380 bps to 450 bps, 400 bps to 430 bps, 30 kDa to 300 kDa molecular weight, 250 kDa to 320 kDa molecular weight, 270 kDa to 300 kDa molecular weight, 4.7 to 46.7 helical turns of duplexed RNA, 30 to 38 helical turns of duplexed RNA, 32 to 36 helical turns of duplexed RNA, and a combination thereof.

In another aspect, Rugged dsRNA is produced by isolating the 5-minute HPLC peak of a tdsRNA preparation.

### 3.3 Rugged dsRNA Preparation

In one embodiment, the starting material for making Rugged dsRNA may be dsRNA prepared in vitro using conditions of this disclosure. For example, the specifically configured dsRNA described in U.S. Pat. Nos. 4,024,222, 4,130,641, and 5,258,369 (which are incorporated by reference herein) are generally suitable as starting materials after selection for rugged dsRNA. tdsRNA (or preparations of tdsRNA) described in this disclosure is also useful as starting material. One starting material may be a preparation of formula A.

After procuring starting material, Rugged dsRNA may be isolated by at least subjecting the partially hybridized strands of a population of dsRNA to conditions that denature most dsRNA (more than 10 wt. % or mol %, more than 20 wt. % or mol %, more than 30 wt. % or mol %, more than 40 wt. % or mol %, more than 50 wt. % or mol %, more than 60 wt. % or mol %, more than 70 wt. % or mol %, more than 80 wt. % or mol %, more than 90 wt. % or mol %, more than 95 wt. % or mol %, or more than 98 wt. % or mol %) in the population, and then selection negatively or positively (or both) for dsRNA that remain partially hybridized. The denaturing conditions to unfold at least partially hybridized strands of dsRNA may comprise an appropriate choice of buffer salts, pH, solvent, temperature, or any combination thereof. Conditions may be empirically determined by observation of the unfolding or melting of the duplex strands of ribonucleic acid. The yield of rugged dsRNA may be improved by partial hydrolysis of longer strands of ribonucleic acid, then selection of (partially) hybridized stands of appropriate size and resistance to denaturation.

The purity of rugged dsRNA, which functions as tdsRNA, may thus be increased from less than about 0.1-10 mol % (e.g., rugged dsRNA is present in at least 0.1 mol % or 0.1 wt percent but less than about 10 mol % or 10 wt percent) relative to all RNA in the population after synthesis to a higher purity. A higher purity may be more than 20 wt. % or mol %, more than 30 wt. % or mol %, more than 40 wt. % or mol %, more than 50 wt. % or mol %, more than 60 wt. % or mol %, more than 70 wt. % or mol %, more than 80 wt. % or mol %, more than 90 wt. % or mol %, more than 98 wt. % or mol %, or between 80 to 98 wt. % or mol %. All wt. % or mol % is relative to all RNA present in the same composition.

Another method of isolating Rugged dsRNA is to employ chromatography. Under analytical or preparative high-performance liquid chromatography, Rugged dsRNA can be isolated from a preparation (e.g., the starting material as described above) to produce poly(I):poly(C₁₂U)ₙ (e.g., poly(I):poly(C₁₁₋₁₄U)ₙ) as a substantially purified and pharmaceutically-active molecule with an HPLC peak of about 4.5 to 6.5 minutes, preferably between 4.5 and 6 minutes and most preferably 5 minutes.

Rugged dsRNA and the method of making rugged dsRNA are described in U.S. Pat. Nos. 8,722,874 and 9,315,538 (incorporated by reference).

### 3.4 Stabilizing Forms

In one embodiment, the tdsRNA may be stabilized by drying to produce dried tdsRNA. Drying may be performed by freeze drying (lyophilization), exposure to a dry gas stream (e.g., nitrogen stream) or exposure to a dry environment. Dried tdsRNA may tdsRNA by itself or tdsRNA in the form of a composition or a medicament (e.g., dried tdsRNA in Water For Injection (WFI), phosphate buffered saline (PBS) or TE buffer).

If a dried tdsRNA is used, the process of administering the tdsRNA may further include the step of reconstituting a dried tdsRNA or a dried composition or dried medicament comprising tdsRNA. The reconstituting step may include the steps of contacting a diluent (e.g., PBS or water) with the dried tdsRNA; and, optionally, agitating the vial containing the dried tdsRNA to dissolve the lyophilized medicament. Contacting a diluent to dried tdsRNA may involve, for example, injecting a diluent into a vial containing lyophilized tdsRNA.

The diluent may be water such as WFI, or it may be water with buffers such as PBS and or TE buffer(e.g., 10 mM Tris, 0.1 mM EDTA, pH 8.0). In a preferred embodiment, nuclease-free water, pH 7.0, can be used for resuspending lyophilized tdsRNA. The use of HPLC-or molecular biology-grade water certified to be RNase-free as a diluent, or as the liquid part of PBS or TE buffer is preferable.

It is preferred that in any embodiment, the diluent, including water or PBS, is nuclease-free and endotoxin-free. Test for nuclease-free water may be by commercially available products such as RNaseAlert® and DNaseAlert™ reagents. Screened for endotoxins may be performed with a Limulus amebocyte lysate (LAL) assay.

In another embodiment, tdsRNA may be stored as an alcohol (e.g., ethanol) precipitate at −80° C. For example, an alcohol precipitate can be made by adding 0.1 volume of 3 M sodium acetate solution to one volume of an RNA solution, mixing, and adding 2.5 to 3 volumes of ethanol and mixing. Then the solution with the precipitate may be chilled to −20° C. or −80° C. for storage.

### 3.5 Stabilizing polymers

In any of the described embodiments, the tdsRNA may be complexed with a stabilizing polymer such as: polylysine, polylysine plus carboxymethylcellulose (lysine carboxy methyl cellulose), polyarginine, polyarginine plus carboxymethylcellulose, or a combination thereof. Some of these stabilizing polymers are described, for example, in U.S. Pat. No. 7,439,349.

### 3.6 Modified Backbone

The tdsRNA may comprise one or more alterations in the backbone of the nucleic acid. For example, configured tdsRNA may be made by modifying the ribosyl backbone of poly(riboinosinic acid) r(Iₙ), for example, by including 2′-O-methylribosyl residues. Specifically configured dsRNA may also be modified at the molecule's ends to add a hinge(s) to prevent slippage of the base pairs, thereby conferring specific bioactivity in solvents or aqueous environments that exist in human biological fluids.

### 4. Additional Agents

The tdsRNA of this disclosure may be in combination with a number of additional agents. Some of the preferred agents are described herein.

### 4.1 Carrier or Vehicle

Suitable agents may include a suitable carrier or vehicle for intranasal mucosal delivery. As used herein, the term “carrier” refers to a pharmaceutically acceptable solid or liquid filler, diluent or encapsulating material. In one aspect, the carrier is a suitable carrier or vehicle for intranasal mucosal delivery.

### 4.2 Absorption-Promoting Agents

Suitable agents may include any suitable absorption-promoting agents. The suitable absorption-promoting agents may be selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacyl methyl sulfoxide, azone (1-dodecylazacycloheptan-2-one or laurocapram), sodium lauryl sulfate, oleic acid, and the bile salts, may be employed to enhance mucosal penetration of the tdsRNA. In additional aspects, surfactants (e.g., polysorbates) are employed as adjunct compounds, processing agents, or formulation additives to enhance intranasal delivery of the tdsRNA.

### 4.3 Delivery-enhancing agents

As used herein, the term “delivery-enhancing agents” refers to any agents which enhance the release or solubility (e.g., from a formulation delivery vehicle), diffusion rate, penetration capacity and timing, uptake, residence time, stability, effective half-life, peak or sustained concentration levels, clearance and other desired intranasal delivery characteristics (e.g., as measured at the site of delivery, or at a selected target site of activity such as the bloodstream) of tdsRNA or other biologically active compound(s).

### 4.4 Mucolytic or Mucus Clearing Agents

In another embodiment, the present compositions may also comprise other suitable agents such as mucolytic and mucus-clearing agents. The term “mucolytic and mucus-clearing agents,” as used herein, refers to any agents which may serve to degrade, thin or clear mucus from intranasal mucosal surfaces to facilitate absorption of intranasally administered biotherapeutic agents including tdsRNA. Based on their mechanisms of action, mucolytic and mucus clearing agents can often be classified into the following groups: proteases (e.g., pronase, papain) that cleave the protein core of mucin glycoproteins, sulfhydryl compounds that split mucoprotein disulfide linkages, and detergents (e.g., Triton X-100, Tween 20) that break non-covalent bonds within the mucus. Additional compounds in this context include, but are not limited to, bile salts and surfactants, for example, sodium deoxycholate, sodium taurodeoxycholate, sodium glycocholate, and lysophosphatidylcholine.

### 4.5 Ciliostatic Agents

In another embodiment, the present compositions may comprise ciliostatic agents. As used herein, the term “ciliostatic agents” refers to any agents which are capable of moving a layer of mucus along the mucosa to removing inhaled particles and microorganisms. Examples of ciliostatic factors include a phenazine derivative, a pyo compound (2-alkyl-4-hydroxyquinolines), and a rhamnolipid (also known as a hemolysin).

### 4.6 Penetration or Permeation-Promoting Agent

In another embodiment, the intranasal mucosal therapeutic and prophylactic formulations of the present disclosure may be supplemented with any suitable penetration-promoting agent that facilitates absorption, diffusion, or penetration of tdsRNA across mucosal barriers. Examples of such agents include sodium salicylate and salicylic acid derivatives (acetyl salicylate, choline salicylate, salicylamide, etc.), amino acids and salts thereof (e.g., monoaminocarboxlic acids such as glycine, alanine, phenylalanine, proline, hydroxyproline, etc., hydroxyamino acids such as serine, acidic amino acids such as aspartic acid, glutamic acid, etc., and basic amino acids such as lysine, etc.—inclusive of their alkali metal or alkaline earth metal salts), and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts).

### 4.7 Vasodilator Agents

In another embodiment, the present formulation may also comprise other suitable agents such as vasodilator agents. Examples of vasodilators include calcium antagonists, potassium channel openers, ACE inhibitors, angiotensin-II receptor antagonists, alpha-adrenergic and imidazole receptor antagonists, beta-1-adrenergic agonists, phosphodiesterase inhibitors, eicosanoids and NO donors.

### 4.8 RNAse Inhibitory Agent and Enzyme Inhibitor

The compositions of the present disclosure may contain an RNase inhibitor or an enzyme inhibitor. Typical enzyme inhibitors that are commonly employed and that may be incorporated into the present disclosure may be, for example, leupeptin, aprotinin, and the like. RNase inhibitors are commonly used as a precautionary measure in enzymatic manipulations of RNA to inhibit and control RNase. These are commercially available from a number of sources such as, for example, Invitrogen (SUPERase, In RNase Inhibitor, RNaseOUT, RNAsecure, and RNase Inhibitor).

### 5. Administration (Delivery)

Administration to the subject or administering to the subject may be in any known form including: systemic administration; intravenous administration; intradermal administration; subcutaneous administration; intramuscular administration; intranasal administration (e.g., pulmonary airway administration); intranasal administration and oral administration at the same time; intraperitoneal administration; intracranial administration; intravesical administration; oral administration (e.g., through the mouth or by breathing through the mouth); topical administration; inhalation administration; aerosol administration; intra-airway administration; tracheal administration; bronchial administration; instillation; bronchoscopic instillation; intratracheal administration; mucosal administration; dry powder administration; spray administration; contact administration; swab administration; intratracheal deposition administration; intrabronchial deposition administration; bronchoscopic deposition administration; lung administration; nasal passage administration; respirable solid administration; respirable liquid administration; dry powder inhalants administration.

The most preferred methods include intranasal administration or intravenous administration. Intranasal administration (sometimes called nasal administration) in this disclosure refers to administering to nasal passages or administering to nasal epithelium.

As another example, administering may be performed by a delivery system or medical device comprising the tdsRNA. The delivery system or medical device may be a nebulizer; a sprayer; a nasal pump; a squeeze bottle; a nasal spray; a syringe sprayer or plunger sprayer(a syringe providing pressure to an attached sprayer or nozzle), a swab; a pipette; a nasal irrigation device; a nasal rinse; or any device for administrating a composition to the inside of the nose.

In contrast, other emulsifying agents typically protect the emulsified droplets by forming a liquid crystalline layer around the emulsified droplets. In compositions that employ a liquid crystalline layer-forming emulsifying agent, the hydrophilic-lipophilic balance (HLB) of the oil phase of the emulsion must be matched with that of the emulsifying agent to form a stable emulsion and, often, one or more additional emulsifying agents (secondary emulsifying agents) must be added to further stabilize the emulsion. The aforementioned liquid crystalline layer also retards the release of the compounds of the dispersed phase upon contact with the target substrate.

The liquid compositions are particularly suited for nasal administration.

### 5.1 Administration Delivery System

Administration may also be from any known delivery system. A delivery system may be at least one selected from the group consisting of: a pill, a capsule, a needle, a cannula, an implantable drug depot, an infusion system (e.g., a device similar to an insulin pump); a nebulizer; a sprayer; a nasal pump; a squeeze bottle; a nasal spray; a syringe sprayer, a plunger sprayer(a syringe providing pressure to an attached sprayer or nozzle); a nasal aerosol device; a controlled particle dispersion device; a nasal aerosol device; a nasal nebulization device; a pressure-driven jet nebulizer; an ultrasonic nebulizer; a breath-powered nasal delivery device; an atomized nasal medication device; an inhaler; a powder dispenser; a dry powder generator; an aerosolizer; an intrapulmonary aerosolizer; a sub-miniature aerosolizer; a propellant based metered-dose inhalers; a dry powder inhalation devices; an instillation device; an intranasal instillation device; an intravesical instillation device; a swab; a pipette; a nasal irrigation device; a nasal rinse; an aerosol device; a metered aerosol device; a pressurized dosage device; a powdered aerosol; a spray aerosol; a spray device; a metered spray device; a suspension spray device; and a combination thereof.

### 5.2 Formulations and Dosage

Formulations for administration (i.e., pharmaceutical compositions) may include a pharmaceutically acceptable carrier with the tdsRNA.

Pharmaceutical carriers include suitable non-toxic vehicles in which a composition of the disclosure is dissolved, dispersed, impregnated, or suspended, such as water, aqueous solutions, or other solvents, fatty materials, celluloses and their derivatives, proteins and their derivatives, collagens, gelatine, polymers, adhesives, sponges, fabrics, and the like and excipients which are added to provide better solubility or dispersion of the drug in the vehicle. Such excipients may include non-toxic surfactants, solubilizers, emulsifiers, chelating agents, binding materials, lubricants, softening agents, and the like. Pharmaceutically acceptable carriers may be, for example, aqueous solutions, syrups, elixirs, powders, granules, tablets, and capsules which typically contain conventional excipients such as binding agents, fillers, lubricants, disintegrants, wetting agents, suspending agents, emulsifying agents, preservatives, buffer salts, flavoring, coloring, and/or sweetening agents.

The tdsRNA may be a combination or any subset of dsRNA described above (e.g., formula A and/or formula B). It is understood that in one aspect, tdsRNA may comprise a combination of all of the examples of tdsRNA described above or any subset of the above examples. With respect to the subsets, the specific exclusion of one or more specific embodiments of tdsRNA is also envisioned. As non-limiting examples, tdsRNA may comprise any of the following: (A) all of formula A and formula B, (B) all of formula A and formula B but without rIₙ·r(C₁₁₋₁₄U)ₙ, (C) formula B, (D) rIₙ·r(C₁₂U)ₙ, (E) formula A but without rIₙ·r(C₁₁₋₁₄U)ₙ, (F) rIₙ·r(C₁₂U)ₙ, and Rugged dsRNA; or(G) rIₙ·r(C₁₁₋₁₄U)ₙ and Rugged dsRNA.

### 5.3 Medicament

In another aspect, a medicament (e.g., a pharmaceutical composition) containing the tdsRNA is provided. Medicaments are pharmaceutically acceptable and may comprise any of the compositions described herein. A medicament may be any of the following, without limitation: aerosols, granules, lyophilized film, powders, solutions, and suspensions; the contents of a sealed container such as, without limitation, an ampule, an intravenous (IV) bag or bottle, or a vial; or the contents of a device such as, without limitation, an implanted depot, an implanted pump, an infusion pump, an inhaler, an insufflator, a mister or a sprayer, a nebulizer, a syringe, or a vaporizer. A use for the medicament may be administering tdsRNA, optional pharmaceutically-acceptable excipients, optional pharmaceutically-acceptable vehicle, and optional pharmaceutically-acceptable carrier by any route such as, but not limited to, at least parenteral administration, mucosal administration, pulmonary administration, or any combination thereof. A hypodermic syringe (e.g., intramuscular, intravenous, subcutaneous), an implanted depot or pump, an infusion pump, an intravenous (IV) drip, or the like may be used for parenteral administration.

The formulation may be a medicament. Medicaments are pharmaceutically acceptable and may comprise any of the compositions described herein. A medicament may be any of the following, without limitation: aerosols, granules, lyophilized film, powders, solutions, and suspensions; the contents of a sealed container such as, without limitation, an ampule, an intravenous (IV) bag or bottle, or a vial; or the contents of a device such as, without limitation, an implanted depot, an implanted pump, an infusion pump, an inhaler, an insufflator, a mister or a sprayer, a nebulizer, a syringe, or a vaporizer. A use for the medicament may be administering tdsRNA, optional pharmaceutically-acceptable excipients, optional pharmaceutically-acceptable vehicle, and optional pharmaceutically-acceptable carrier by any route such as, but not limited to, at least parenteral administration, mucosal administration, pulmonary administration, or any combination thereof. A hypodermic syringe (e.g., intramuscular, intravenous, subcutaneous), an implanted depot or pump, an infusion pump, an intravenous (IV) drip, or the like may be used for parenteral administration.

### 5.4 Dosage for the Average Subject

The dosages are generally applicable to a subject as described in another section of this disclosure. In a preferred embodiment, the subject is human.

For a subject (especially human) the dose of tdsRNA for iv administration may be: 0.1 μg to 1,200 mg; 0.1 to 25 mg; 25 mg to 50 mg; 50 mg to 100 mg; 100 mg to 200 mg; 200 mg to 400 mg; 400 mg to 800 mg; 800 mg to 1,200 mg. For example, iv dosages may be 25 mg; 50 mg; 125 mg; 200 mg; 250 mg; 400 mg; 500 mg; 800 mg; 1,000 mg; 1,200 mg. In a preferred embodiment, the tdsRNA may be administered two times a week and at 400 mg per administration. In another preferred embodiment, the tdsRNA may be administered two times a week at 200 mg per administration (400 mg/week or an average dose of 57 mg/day) for the first 2 weeks, two times a week at 400 mg per administration (800 mg/week or an average dose of 114 mg/day) after the first 2 weeks.

For intranasal dosage, the dose of tdsRNA may be: 0.1 μg to 1,200 μg; 0.1 to 25 μg; 25 μg to 50 μg; 50 μg to 100 μg; 100 μg to 200 μg; 200 μg to 400 μg; 400 μg to 800 μg; 800 μg to 1,250 μg. For example, iv dosages may be 25 μg; 50 μg; 125 μg; 250 μg; 500 μg; 1,000 μg; 1,250 μg.

### 5.5 Amount Per Unit Dose

The amount per unit dose of tdsRNA may be at least one selected from 0.1 mg/kg, 0.2 mg/kg, 0.4 mg/kg, 0.6 mg/kg, 0.8 mg/kg, 1 mg/kg, 2 mg/kg, 3 mg/kg, 4 mg/kg, 5 mg/kg, 6 mg/kg, 8 mg/kg, 10 mg/kg.

### 5.6 Specific Examples

In one embodiment, the tdsRNA is administered iv at a dose from about 1 mg/kg to 10 mg/kg biweekly. As another example, the administration may be in 50-1400 milligrams every other day, leading to an average daily dosage of 25-700 milligrams per day. In one embodiment, the tdsRNA is administered at a dose from about 0.50 mg/kg to 10 mg/kg every other week. 50-1400 milligrams every other day, leading to an average daily dosage of 25-700 milligrams per day.

### 5.7 Dose Frequency

In certain embodiments, the tdsRNA is administered at a frequency selected from the group consisting of: one dose per day, one dose every 2 days, one dose every 3 days, one dose every 4 days, one dose every 5 days, 4 doses a week, 3 doses a week, 2 doses a week, 1 dose a week, one dose every two weeks, one dose every three weeks, one dose every four weeks, and one dose every month. Nasal administration may be as listed above or may be 2 doses per day or three doses per day. Administration or dosing can be continued as long as they have a beneficial effect on the subject. One preferred dosage is 200 mg or 400 mg, twice a week for a total of 400 mg or 800 mg a week.

### 6. Nasal Administration Devices

A device or delivery system, encompassing a composition of the disclosure is also an embodiment.

The composition of the disclosure may be delivered by any nasal administration device or combination of devices. A combination refers to a composition that is both administered by two different devices or a device having the feature of two devices. Non-limiting examples of suitable devices that can be use individually or together include at least one selected from the group consisting of: a nebulizer; a sprayer(e.g., a spray bottle such as “Nasal Spray Pump w/Safety Clip, Pfeiffer SAP #60548; a squeeze bottle (e.g., bottle commonly used for nasal sprays, including ASTELIN (azelastine hydrochloride, Medpointe Healthcare Inc.) and PATANASE (olopatadine hydrochloride, Alcon, Inc.); a nasal pump spray (e.g., APTAR PHARMA nasal spray pump); a controlled particle dispersion devices (e.g., VIANASE electronic atomizer); a nasal aerosol device (e.g., ZETONNA nasal aerosol); a nasal nebulization device (e.g., EASYNOSE nebulizer, a pressure-driven jet nebulizer, or an ultrasonic nebulizer); a powder nasal delivery devices (e.g., OPTINOSE breath-powered nasal delivery device); an atomized nasal medication device (e.g., LMA MAD NASAL device); an instillation device; an inhalation device (e.g., an inhaler); a powder dispenser; a dry powder generator; an aerolizer(e.g., intrapulmonary aerosolizer or a sub-miniature aerosolizer, metered aerosol, powdered aerosol, spray aerosol); a spray; a metered spray; a metered dose inhalers (e.g., a propellant based metered-dose inhaler); a dry powder inhalation device; an intranasal instillation device; an intravesical instillation device; an insufflation device.

An application device for application to mucous membranes, such as, that of the nose, throat, and/or bronchial tubes (i.e., inhalation). This can be a swab, a pipette or a device for nasal irrigation, nasal rinse, or nasal lavage.

Another example is a syringe or plunger-activated sprayer. This could be, for example, a sprayer head (or nozzle) attached, for example, via a Luer lock, to a syringe. The syringe applies pressure to a composition that flows through the sprayer head and produces a spray or an aerosol.

### 7. Discussion of Further Embodiments and Features

### 7.1 Subject or Patient

As used herein, a “subject” has the same meaning as a “patient” and is a mammal, preferably a human. In addition to humans, categories of mammals within the scope of the present disclosure include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc. Other examples of subjects include any animal such as civet cats, swine, cattle, horses, camels, cats, dogs, rodents, birds, bats, rabbits, ferrets, mink, and the like. As used herein, the terms “patient” or “subject” are used interchangeably.

### 7.2 Devices and Kits

In another aspect, the present disclosure relates to and comprises a therapeutic device for intranasal delivery. In one embodiment, the therapeutic device may comprise any suitable devices charged with a preparation of tdsRNA.

### 7.3 Effective Amount: Therapeutically or Prophylactically Effective Amount

The compositions are delivered in effective amounts. The term “effective amount” refers to the amount necessary or sufficient to realize a desired biological effect which is, for example, reducing, stopping the advance of, or reversing the symptoms of PCC of fatigue. In addition to the sample dosages and administration methods mentions, one of ordinary skill in the art can empirically determine the effective amount of the tdsRNA without necessitating undue experimentation. It is preferred that a maximum dose be used, that is, the highest safe dose according to medical judgment.

Effective dosage forms, modes of administration, and dosage amounts may be determined empirically, and making such determinations is within the skill of the art. It is understood by those skilled in the art that the dosage amount will vary with the route and mode of administration, the rate of excretion, the duration of the treatment, the identity of any other drugs (e.g., antiviral agent) being co-administered, the age, size, species of mammal (e.g., human patient), and other factors well known in the arts of medicine and veterinary medicine. In general, a suitable dose of any active agent disclosed herein or a composition containing the same will be that amount of the active agent (tdsRNA) or composition comprising the active agent, which is the lowest dose effective to produce the desired effect. The desired effect may be to reduce the severity or duration of a symptom of a viral infection or PCC of fatigue.

### 8. Other Aspects

In this specification, stating a numerical range, it should be understood that all values within the range are also described (e.g., one to ten also includes every value between one and ten as well as all intermediate ranges such as two to ten, one to five, and three to eight). The term “about” may refer to the statistical uncertainty associated with a measurement or the variability in a numerical quantity that a person skilled in the art would understand does not affect the operation of the disclosure or its patentability.

All modifications and substitutions that come within the meaning of the claims and the range of their legal equivalents are to be embraced within their scope. A claim which recites “comprising” allows the inclusion of other elements to be within the scope of the claim. The disclosure is also described by such claims reciting the transitional phrases “consisting essentially of” (i.e., allowing the inclusion of other elements to be within the scope of the claim if they do not materially affect the operation of the disclosure) or “consisting of” (i.e., allowing only the elements listed in the claim other than impurities or inconsequential activities which are ordinarily associated with the disclosure) instead of the “comprising” term. Any of these three transitions can be used to claim the disclosure.

An element described in this specification should not be construed as a limitation of the claimed disclosure unless it is explicitly recited in the claims. Thus, the granted claims are the basis for determining the scope of legal protection instead of a limitation from the specification which is read into the claims. In contradistinction, the prior art is explicitly excluded from the disclosure to the extent of specific embodiments that would anticipate the claimed disclosure or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements, embodiments, and aspects disclosed herein are also considered to be aspects and embodiments of the disclosure. Similarly, generalizations of the disclosure's description are considered to be part of the disclosure.

From the foregoing, it would be apparent to a person of skill in this art that the disclosure can be embodied in other specific forms without departing from its spirit or essential characteristics.

While the disclosure has been described in connection with what is presently considered to be the most practical and preferred embodiment, it is to be understood that the disclosure is not to be limited to the disclosed embodiment, but on the contrary, is intended to cover various modifications and equivalent arrangements included within the spirit and scope of the appended claims.

### 9. Incorporation by Reference

All publications, patent applications, and patents mentioned herein are hereby incorporated by reference in their entirety as if each individual publication or patent was specifically and individually indicated to be incorporated by reference. These patents include, at least, U.S. Pat. Nos. 4,024,222, 4,130,641, 5,258,369,7,439,349, 8,722,874 and 9,315,538. In case of conflict, the present application, including any definitions herein, will control.

### EXAMPLES

### Example 1 Nasal Administration of tdsRNA

Rintatolimod is a well-defined selective Toll-like receptor 3 (TLR3) agonist inducing innate immune antiviral responses. Rintatolimod has been administered intravenously in approximately 100,000 doses in clinical trials and compassionate use programs. Besides, intranasal administration of rintatolimod as a universal flu adjuvant was found to be well tolerated.

A phase I trial was performed to assess the safety, tolerability and biological activity of repeated administration of rintatolimod intranasally every other day for 13 days (7 doses) in healthy volunteers. Also, this study was performed for the further development of rintatolimod as a potential treatment modality for COVID-19 and other pulmonary viral diseases.

This experiment was performed to determine the safety and possible efficacy of intranasal administration of tdsRNA. This study showed that rintatolimod, a form of tdsRNA, is well tolerated when administered intranasally at dose levels up to 1250 μg every other day for seven doses over 13 days. There were no severe adverse events with the described dosage.

### Example 2 A Stable Form of tdsRNA

To determine if tdsRNA is stable at room temperature after drying, we analyzed the stability of rintatolimod in PBS. Briefly, rintatolimod in PBS was lyophilized and stored at room temperature for 3 months. After storage the rintatolimod was reconstituted with water and examined for activity and structure. No difference in activity or structure was observed.

### Example 3 A Study Of tdsRNA for Treating Patients With PCC of Fatigue

In this study, subjects who have PCC of fatigue were treated to see if they would benefit from tdsRNA therapy. Each of the patients treated have had COVID-19 (i.e., SARS-COV-2 infection) and had their SARS-COV-2 infections resolved. That is, at the time of the study, there should be no detectable levels of SARS-COV-2 in the subject using nucleic acid (e.g., RT-PCR), antibody and protein-based detection methods. Evaluation of safety is a secondary objective in this study and no serious adverse events were observed.

This ongoing study is a prospective, open-label, multicenter study to treat PCC of fatigue patients with tdsRNA (poly I:poly C₁₂U, also called rintatolimod). During the study, subjects are evaluated at baseline (Week 0) using a PCC of fatigue Instrument. They then begin treatment with tdsRNA and the PCC of fatigue symptoms (e.g., difficulty concentrating or focusing) is evaluated over time.

Dosing Schedule: Patients received open label intravenous (IV) rintatolimod twice weekly. During the first two (2) weeks (with dosing twice a week) the patients receive four 200 mg (80 ml) doses of rintatolimod. If this dose is well-tolerated, the dose will be increased at week 3 (dose 5) to 400 mg (160 ml). The first infusion at each dose level is given over 60±5 minutes; if well tolerated without any significant infusion-related side effects (e.g., flu-like symptoms), the next infusion will be over 45±5 minutes; if well tolerated, all remaining infusions will be given over 35±5 minutes. Rintatolimod is provided in glass vials containing 200 mg (80 ml; concentration of 2.5 mg/ml) as a solution for intravenous infusion which is stored at temperatures from 2° C. to 8° C.

Results at 14 weeks:

Our studies are ongoing and our initial findings of the first two enrolled patients after 14 weeks are shown below in Table 1 where the data is based on patient responses to the PCC of fatigue Instrument.

The results seen in the first two patients are very dramatic. PCC of fatigue symptom severity is as follows: 0 is none. 1 to 3 is considered mild. 4 to 6 is considered moderate and 7 to 10 is considered severe. As can be seen in the data, by weeks 11-14, there was a clinically significant decrease in difficulty concentrating or focusing. That is both patients had improvement in their ability to concentrate or focus (see, Difficulty Concentrating or Focusing scores in Table 1).

TABLE 1
PCC of fatigue Symptoms Over Time

| Symptoms | Patient # | Week 0 | Week 4 | Week 9 | Week 11 | Week 14 |
|---|---|---|---|---|---|---|
| Difficulty | #1 | 6.0 | 4.0 | 3.0 | 2.0 | 2.0 |
| Concentrating | | | | | | |
| or Focusing | #2 | 5.5 | 4.0 | 1.0 | 1.0 | 1.0 |

The results clearly show a significant reduction in neurocognitive symptoms associated with PCC of fatigue, in patients after the administration of rintatolimod. The symptom of difficulty concentrating or focusing was reduced in severity from 5.5 and 6.0 at baseline to 1.0 and 2.0 at Weeks 11-14. In addition, the rintatolimod treatments were well-tolerated and there were no severe adverse events.

### Example 4 A Study of tdsRNA for Treating Patients With PCC of Fatigue

In this study, PCC of fatigue patients were tested to see if they would benefit from tdsRNA treatment. Evaluation of safety is a secondary objective in this study and no serious adverse events were observed.

This is a prospective, open-label, multi-center study to treat PCC of fatigue patients with tdsRNA (e.g., Ampligen®). Baseline period will be up to twelve (12) weeks and patients will be treated: 1) for at least a minimum of 24 weeks or as long as the patient is clinically benefiting from the treatment or 2) until dose-limiting toxicity, if any, occurs. At this time, there is no indication of toxicity.

While parts of Example 4 is written in the present tense or future tense, it should be understood that this study is ongoing and at least 4 patients have been studied for at least 24 weeks after the first administration of rintatolimod and additional patients in the study is being added and monitored.

Several quality of life and activity measures including Karnofsky Performance Scores, and PCC of fatigue symptoms were monitored through the study.

Dosing Schedule: Patient received open label intravenous (IV) poly I:poly C₁₂U (Ampligen®) twice weekly for at least 24 weeks. During the first two (2) weeks (doses 1 through 4) the patient received 200 mg (80 ml) doses of poly I:poly C₁₂U (Ampligen®). The dose is increased at week 3 (dose 5) to 400 mg (160 ml). Dosages will be given between 30 to 60 minutes depending on patient tolerance. This dosage schedule is a preferred embodiment of all the claims in this disclosure.

### Evaluations and Assessments

Statistical evaluation at the endpoint is performed for(1) Karnofsky Performance Score (KPS) and PCC of fatigue symptoms.

### Karnofsky Performance Scale and Procedures

### General Procedures for Determining Karnofsky Performance Scores (KPS).

The KPS is a global evaluation of the patient's ability to conduct daily activities, including work activities and self-care activities. The KPS is sensitive to effective therapeutic intervention in chronic disease states. Following two (2) assessments at baseline, a KPS is obtained at week 8, week 16, week 24 and every 12 weeks thereafter during the study, based upon a questionnaire (attached) completed by the patient and an interview which includes discussion of specific signs and symptoms, basic functional accomplishments (e.g., daily care activities), changes in activities and changes in medications taken. In addition, the assignment of the score may include discussions with a significant other(i.e., spouse, companion, or custodian needed to care for patient's daily needs). The KPS score will be assigned by the principal investigator or in (his/her) absence by only one additional designated individual at each site.

Karnofsky Performance Scale is assigned as follows: 100 Normal activity; no complaints; no evidence of disease. 90 Able to carry on normal activity; minor signs or symptoms of disease. 80 Normal activity with effort; some signs and symptoms of disease. 70 Cares for self, unable to carry on normal activity or do active work. 60 Requires occasional assistance but is able to care for most of needs. 50 Requires considerable assistance for daily care. 40 Disabled; unable to care for self, requires special care and assistance. 30 Severely disabled, bedridden although death is not imminent. 20 Very sick; hospitalization and/or nursing care is necessary; active supportive treatment is necessary. 10 Moribund; fatal processes progressing rapidly. 0 Dead.

### PCC of Fatigue Symptoms Monitored

PCC of fatigue symptoms monitored in this study include one or more of mental fatigue, difficulty concentrating or focusing, and post exertional malaise (tiredness the day after exercise).

### Results:

Our studies are ongoing. Our initial findings after 12 weeks of tdsRNA (rintatolimod) are shown below in Table 2.

PCC of fatigue severity is as follows: 0 is none. 1 to 3 is considered mild. 4 to 6 is considered moderate and 7 to 10 is considered severe. Baseline questions were established with values greater than or equal to=2. The initial dose was 200 mg per dose administered twice weekly (400 mg/subject/week) on weeks 1 and 2. The doses escalated to 400 mg per dose administered twice weekly (800 mg/subject/week) from week 3, and the current dosage, 12 weeks into the study, is 400 mg per dose administered twice weekly (800 mg/subject/week).

TABLE 2
Summary Of Patient Symptoms Over Time
Showing Significant Improvement

| | | | Difficulty | |
|---|---|---|---|---|
| | Inability to | | concen- | AVERAGE |
| PCC of fatigue | exercise or | Mental | trating or | ALL |
| Symptoms | be active | Fatigue | focusing | SYMPTOMS |
| BSL 1 (−2 weeks) | 10 | 9 | 5 | |
| BSL 2 (−1 week) | 10 | 10 | 7 | |
| BSL Avg | 10.00 | 9.50 | 6.00 | 8.50 |
| Week 1 | 10 | 7 | 3 | |
| Week 2 | 10 | 7 | 2 | |
| Week 3 | 10 | 7 | 4 | |
| Week 4 | 10 | 9 | 7 | |
| Avg. Weeks 1-4 | 10.00 | 7.50 | 4.00 | 7.17 |
| Week 5 | 10 | 8 | 6 | |
| Week 6 | 10 | 8 | 8 | |
| Week 7 | 10 | 8 | 8 | |
| Week 8 | 10 | 8 | 8 | |
| Avg. Weeks 5-8 | 10.00 | 8.00 | 7.50 | 8.50 |
| Week 9 | 10 | 7 | 3 | |
| Week 10 | 10 | 7 | 2 | |
| Week 11 | 9 | 7 | 2 | |
| Week 12 | 7 | 6 | 3 | 3 |
| Avg. Weeks 9-12 | 9.00 | 6.75 | 2.50 | 6.08 |
| Change after 12 | −1.00 | −2.75 | −3.50 | −2.42 |
| weeks | | | | |

Statistical analysis was performed on the data above and this analysis is shown in Table 3 below.

TABLE 3
Statistical Analysis of Symptom Improvement at Week 12
XLSTAT 2020.5.1.1078 - Comparison of two samples (Wilcoxon, Mann-Whitney, . . . )

Sample 1: Workbook = Patient SNV-288 PCC of fatigue Questionnaire sort″!$D$7:$D$18/11 rows and 1 column
Sample 2: Workbook = Patient SNV-288 PCC of fatigue Questionnaire sort″!$s$7:$s$18/11 rows and 1 column
Hypothesized difference (D): 0
Significance level (%): 5
p-value: Asymptotic p-value
Continuity correction: Yes
Summary statistics: PCC of fatigue Questionnaire Baseline to [AVERAGE] Week 12 Value
(Patient SNV-288)
11 Symptoms with Baseline value >/=2

| | | Obs. with | Obs. without | | | | Std. |
|---|---|---|---|---|---|---|---|
| Variable | Observations | missing data | missing data | Minimum | Maximum | Mean | deviation |
| BSL Avg | 11 | 0 | 11 | 2.000 | 10.000 | 4.727 | 2.805 |
| Avg. Weeks 9-12 | 11 | 0 | 11 | 0.250 | 9.000 | 2.705 | 2.919 |
| Mann-Whitney test/Two-tailed test: | | | | | | | |
| U | 91 | | | | | | |
| U (standardized) | 1.976 | | | | | | |
| Expected value | 60.500 | | | | | | |
| Variance (U) | 230.476 | | | | | | |
| p-value (Two-tailed) | 0.048 | | | | | | |
| alpha | 0.050 | | | | | | |

An approximation has been used to compute the p-value.
The continuity correction has been applied.
Test interpretation:
H0: The difference of location between the samples is equal to 0.
Ha: The difference of location between the samples is different from 0.
As the computed p-value is lower than the significance level alpha = 0.05, one should reject the null hypothesis H0, and accept the alternative hypothesis Ha.
Ties have been detected in the data and the appropriate corrections have been applied.

Based on P-value (Two-tailed) of less than 0.05, the results show a significant reduction in PCC of fatigue symptoms after a 12-week treatment.

Our studies have been extended to 24 weeks with a total of 4 patients. Our findings are listed below. The findings for these four enrolled patients are shown below.

The reduction in mental fatigue observed in the first four patients provide preliminary evidence of Ampligen's effect in the Post-COVID Condition of fatigue. Post-COVID Conditions symptom severity score is as follows: 0 is none, 1 to 3 is considered mild, 4 to 6 is considered moderate, and 7 to 10 is considered severe. As shown from these data, by week 12, compared to baseline, there was a clinically significant decrease in the mental fatigue-related questions (see Tables below). The data up to 24 weeks is shown below:

TABLE 4A
Data of Sub-set of 3 Questions from Patient Questionnaires (Patient 1)
Patient 1

| | Date of Visit | | | | | |
|---|---|---|---|---|---|---|
| | Day | Day | | Week | Week | |
| | −12 | 0 | | 12 | 24 | Change |
| | PCC of fatigue Symptoms* | | | | | |
| | BSL | BSL | BSL | Week | Week | Week |
| | 1 | 2 | Avg | 12 | 24 | 24 |
| Inability to exercise or be | 10 | 10 | 10 | 7 | −1 | −3 |
| active* | | | | | | |
| Mental Fatigue* | 10 | 8 | 9 | 7 | 7 | −2 |
| Post exertional malaise | 8 | 8 | 8 | 5 | 4 | −4 |
| (tiredness the day after | | | | | | |
| exercise)* | | | | | | |
| Average of All Symptoms | | | 9 | | | −3 |

TABLE 4B
Data of Sub-set of 3 Questions from Patient Questionnaires (Patient 2)
Patient 2

| | Date of Visit | | | | | |
|---|---|---|---|---|---|---|
| | Day | Day | | Week | Week | |
| | −12 | 0 | | 12 | 24 | Change |
| | PCC of fatigue Symptoms* | | | | | |
| | BSL | BSL | BSL | Week | Week | Week |
| | 1 | 2 | Avg | 12 | 24 | 24 |
| Inability to exercise or be | 7 | 9 | 8 | 4 | 1 | −7 |
| active* | | | | | | |
| Mental Fatigue* | 6 | 6 | 6 | 3 | 1 | −5 |
| Post exertional malaise | | 6 | 6 | 1 | 1 | −5 |
| (tiredness the day after | | | | | | |
| exercise)* | | | | | | |
| Average of All Symptoms | | | 6.7 | | | −6 |

TABLE 4C
Data of Sub-set of 3 Questions from Patient Questionnaires (Patient 3)
Patient 3

| | Date of Visit | | | | | |
|---|---|---|---|---|---|---|
| | Day | Day | | Week | Week | |
| | −7 | 0 | | 12 | 24 | Change |
| | PCC of fatigue Symptoms* | | | | | |
| | BSL | BSL | BSL | Week | Week | Week |
| | 1 | 2 | Avg | 12 | 24 | 24 |
| Inability to exercise or be | 10 | 10 | 10 | 7 | 7 | −3 |
| active* | | | | | | |
| Mental Fatigue | 9 | 10 | 9.5 | 6 | 6 | −4 |
| Post exertional malaise | 6 | 9 | 7.5 | 2 | 7 | −1 |
| (tiredness the day after | | | | | | |
| exercise)* | | | | | | |
| Average of All Symptoms | | | 9 | | | −2.3 |

TABLE 4D
Data of Sub-set of 3 Questions from Patient Questionnaires (Patient 4)
Patient 4

| | Date of Visit | | | | | |
|---|---|---|---|---|---|---|
| | Day | Day | | Week | Week | |
| | −13 | 0 | | 12 | 24 | Change |
| | PCC of fatigue Symptoms* | | | | | |
| | BSL | BSL | BSL | Week | Week | Week |
| | 1 | 2 | Avg | 12 | 24 | 24 |
| Inability to exercise or be | 9 | 9 | 9 | 8 | 7 | −2 |
| active* | | | | | | |
| Mental Fatigue* | 7 | 9 | 8 | 7 | 7 | −1 |
| Post exertional malaise | 7 | 9 | 8 | 9 | 8 | 0 |
| (tiredness the day after | | | | | | |
| exercise)* | | | | | | |
| Average of All Symptoms | | | 8.3 | | | −1 |

*For Table 4A, 4B, 4C, and 4D, the following applies: For the inability to exercise or be active or the mental fatigue symptoms: Severity: 0=None; 1-3=Mild; 4-6=Moderate; 7-10=Severe; 9-10=Very Severe. For the post exertional malaise (tiredness the day after exercise) symptom: 1-3=Mild; 4-6=Moderate; 7-8=Severe; 9-10=Very Severe. p-value calculated using above data from 4 patients, 3 questions, BSL compared to Week 12 and 24 values is as follows: In a Mann-Whitney (Two-tailed test), with N=4 patients, the P value for 10 Week 12 is 0.002 and the P value for week 24 is 0.001. The raw data and statistical analysis from which leads to Table 5 above is shown and discussed below (see, e.g., Tables 5, 6 and 7).

TABLE 5
Data of Baseline & Week 24 Values Mental Fatigue
Symptoms in PCC of fatigue Patients

| PCC of fatigue Symptoms | Patient | BSL Avg | Wk 12 | Wk 24 |
|---|---|---|---|---|
| Inability to exercise or be active | 1 | 10.0 | 7.0 | 7.0 |
| Mental Fatigue | 1 | 9.0 | 7.0 | 7.0 |
| Inability to exercise or be active | 2 | 8.0 | 4 | 1 |
| Mental Fatigue | 2 | 6.0 | 3 | 1 |
| Inability to exercise or be active | 3 | 10.0 | 7.0 | 7.0 |
| Mental Fatigue | 3 | 9.5 | 6.0 | 6.0 |
| Inability to exercise or be active | 4 | 9.0 | 8.0 | 7.0 |
| Mental Fatigue | 4 | 8.0 | 7.0 | 7.0 |
| Post exertional malaise | 1 | 8.0 | 5.0 | 4.0 |
| (tiredness the day after exercise) | 2 | 6.0 | 1.0 | 1.0 |
| | 3 | 7.5 | 2.0 | 7.0 |
| | 4 | 8.0 | 9.0 | 8.0 |
| Average | | 8.25 | 5.50 | 5.25 |
| Median | | 8.0 | 6.5 | 7.0 |
| Mann-Whitney (4 Patients) | | | 0.002 | 0.001 |
| p-value (Two tailed) | | | | |

Sub-set of 3 Questions from Patient Questionnaires
Severity: 0 = None, 1 to 3 = Mild, 4 to 6 = Moderate, 7 to 10 = Severe

TABLE 6
Post-COVID Condition of Fatigue Over Time, Comparison of Week 24 to Baseline
P-Value Statistical Output
Summary statistics: Comparison of Week 24 to Baseline

| | | Obs. | Obs. | | | | |
|---|---|---|---|---|---|---|---|
| | | with | without | | | | |
| | | missing | missing | | | | Std. |
| Variable | Observations | data | data | Minimum | Maximum | Mean | deviation |
| Baseline Average | 12 | 0 | 12 | 6.000 | 10.000 | 8.250 | 1.340 |
| Week 24 value | 12 | 0 | 12 | 1.000 | 8.000 | 5.250 | 2.734 |
| Mann-Whitney test/Two-tailed test: | | | | | | | |
| U | 126 | | | | | | |
| U (standardized) | 0.000 | | | | | | |
| Expected value | 72.000 | | | | | | |
| Variance (U) | 291.522 | | | | | | |
| p-value (Two-tailed) | 0.001 | | | | | | |
| Alpha | 0.050 | | | | | | |

The p-value is computed using an exact method. Time elapsed: 0 s.
Test interpretation:
H0: The difference of location between the samples is equal to 0.
Ha: The difference of location between the samples is different from 0.
As the computed p-value is lower than the significance level alpha = 0.05, one should reject the null hypothesis H0, and accept the alternative hypothes is Ha.
Ties have been detected in the data and the appropriate corrections have been applied.

TABLE 7
Post-COVID Condition of Fatigue Over Time
P-Value Statistical Output
Summary statistics: Comparison of Week 12 to Baseline

| | | Obs. | Obs. | | | | |
|---|---|---|---|---|---|---|---|
| | | with | without | | | | |
| | | missing | missing | | | | Std. |
| Variable | Observations | data | data | Minimum | Maximum | Mean | deviation |
| Baseline Average | 12 | 0 | 12 | 6.000 | 10.000 | 8.250 | 1.340 |
| Week 12 Value | 12 | 0 | 12 | 1.000 | 9.000 | 5.500 | 2.505 |
| Mann-Whitney test/Two-tailed test: | | | | | | | |
| U | 122 | | | | | | |
| U (standardized) | 0.000 | | | | | | |
| Expected value | 72.000 | | | | | | |
| Variance (U) | 294.913 | | | | | | |
| p-value (Two-tailed) | 0.002 | | | | | | |
| Alpha | 0.050 | | | | | | |

The p-value is computed using an exact method. Time elapsed: 0 s.
Test interpretation:
H0: The difference of location between the samples is equal to 0.
Ha: The difference of location between the samples is different from 0.
As the computed p-value is lower than the significance level alpha = 0.05, one should reject the null hypothesis H0, and accept the alternative hypothesis Ha.
Ties have been detected in the data and the appropriate corrections have been applied.

The results of some individual symptoms related to PCC of fatigue are very dramatic. As can be seen in the data, by week 12, there was a significant decrease in the adverse symptoms of PCC of fatigue. Comparing BSL average from before treatment to the values at 12 weeks, we see some significant improvements. For example, inability to exercise or be active was reduced from 10 to 7; Mental Fatigue was reduced from 9.5 to 6; and difficulty concentrating or focusing was reduced from 6 to 3. This data clearly show that administration of tdsRNA (Ampligen®) has a beneficial effect on reducing PCC of fatigue symptom.

As stated above, the data has been extended to 24 weeks of therapy with 4 participants/subjects are equally dramatic. Before discussing the results, it is noted that the value of the measurement, as stated in the Tables above, is a measurement of impairment. Therefore, the best result is a value of zero or 1—meaning no or minor impairment, respectively. Also it follows that a lower value is considered to be better for the patient.

In the PCC of fatigue Questionnaire which is collected weekly, the severity is defined as 0 is none; 1 to 3 is mild; 4 to 6 is moderate; 7 to 10 is severe. In the Specific Symptom Severity Questionnaire, collected every 12 weeks, severity is defined as 1 to 3 is mild; 4 to 6 is moderate; 7 to 8 is severe, and 9-10 is very severe.

For patient 1, the “inability to exercise or be active” value decreased from a baseline average of 10, to a 12 week value of 7 and a 24 week value of 7; the “mental fatigue” value decreased from a baseline average of 9 to a 12 week value of 7 and a 24 week value of 7; and the “post exertional malaise (tiredness the day after exercise)” value decreased from a baseline average of 8 to a 12 week value of 5 and a 24 week value of 4.

For patient 2, the “inability to exercise or be active” value decreased from a baseline average of 8, to a 12 week value of 4, and a 24 week value of 1; the “mental fatigue” value decreased from a baseline average of 6 to a 12 week value of 3 and a 24 week value of 1; and the “post exertional malaise (tiredness the day after exercise)” value decreased from a baseline average of 6 to a 12 week value of 1 and a 24 week value of 1.

For patient 3, the “inability to exercise or be active” value decreased from a baseline average of 10, to a 12 week value of 7 and a 24 week value of 7; the “mental fatigue” value decreased from a baseline average of 9.5, to a 12 week value of 6 and a 24 week value of 6; and the “post exertional malaise (tiredness the day after exercise)” value changed from a baseline average of 7.5 to a 12 week value of 2 and a 24 week value of 7.

For patient 4, the “inability to exercise or be active” value decreased from a baseline average of 9, to a 12 week value of 8 and a 24 week value of 7; the “mental fatigue” value decreased from a baseline average of 8 to a 12 week value of 7 and a 24 week value of 7; the “post exertional malaise (tiredness the day after exercise)” value was 8 at baseline, 9 at 12 weeks and 8 at 24 weeks.

The data show that for some patients the results are quite dramatic. For example, for patient 2, the average change for all symptoms discussed in Tables 4-7 (i.e., the “inability to exercise or be active;” “mental fatigue;” and “post exertional malaise (tiredness the day after exercise)”) was about −6 (avg of 8 to 1, 6 to 1 and 6 to 1, a negative value means an improvement in the patient and a decrease in the severity of a symptom) and after 24 weeks the patient has significantly improved and has only limited and minimal disability in each of the categories with a value of 1. Other patients such as patient 1, patient 3 and patient 4 did not recover quite as dramatic but each of them nevertheless has reduced disability in each of the measurements. The decrease in the average measurements for symptoms discussed in Tables 4-7 (i.e., the “inability to exercise or be active;” “mental fatigue;” and “post exertional malaise (tiredness the day after exercise)”) for patients 1, 3 and 4 are −3, −2.33 and−1 respectively. Therefore, in each case, improvements were seen at least by 12 weeks and continued at 24 weeks.

1. A method for treating a subject that has previously been infected with SARS-COV-2 and exhibiting at least one Post COVID-19 Conditions of fatigue (PCC of fatigue) symptom comprising the steps of:
administering to the subject a therapeutically effective amount of a composition comprising therapeutic double-stranded RNA (tdsRNA),
wherein the tdsRNA is at least one selected from the group consisting of
rIₙ·r(CₓU)ₙ (formula A); and
rugged dsRNA (formula B);
wherein x is at least one selected from the group consisting of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 4-29, 4-30, 14-30, 15-30, 11-14, and 30-35.

2. The method of claim 1, further comprising
a) an optional determining step (a) before the administering step, the determining step comprising determining that the subject was previously infected with SARS-COV-2 and performing the administering step if the subject was previously infected with SARS-COV-2; and
b) an optional determining step (b) before the administering step, the determining step comprising determining the subject has no history of chronic fatigue or chronic fatigue symptoms before being infected by SARS-COV-2 and performing the administering step if the subject has no history of chronic fatigue or chronic fatigue symptoms;
wherein step a) and/or step b) may be performed in any order before the administering step.

3. The method of claim 1,
wherein the subject exhibits at least one, at least two, at least three, or at least four PCC of fatigue symptom selected from the group consisting of: difficulty concentrating or focusing, inability to exercise or be active, mental fatigue, and post exertional malaise (tiredness the day after exercise), wherein the method reduces at least one PCC of fatigue symptom.

4. The method of claim 1, wherein the subject is a mammal, preferably a host of SARS-COV-2 such as human ACE2 receptor expressing mouse; more preferably a hamster, such as Mesocricetus auratus or Cricetulus griseus, or a monkey, such as Macaca mulatta or Macaca fascicularis; and most preferably a human.

5. The method of claim 1,
wherein at least 90 wt. % of the tdsRNA is larger than a size selected from the group consisting of: 40 basepairs; 50 basepairs; 60 basepairs; 70 basepairs; 80 basepairs; and 380 basepairs; or
wherein at least 90 wt. % of the tdsRNA is smaller than a size selected from the group consisting of: 50,000 basepairs; 10,000 basepairs; 9000 basepairs; 8000 basepairs; 7000 basepairs; and 450 basepairs.

6. The method of claim 1, wherein n is a number with a value selected from the group consisting of: 40 to 50,000; 40 to 40,000; 50 to 10,000; 60 to 9000; 70 to 8000; 80 to 7000; and 380 to 450.

7. The method of claim 1,
wherein n is from 40 to 40,000;
wherein the tdsRNA has about 4 to about 4000 helical turns of duplexed RNA strands; or
wherein the tdsRNA has a molecular weight selected from the group consisting of: 2 kDa to 30,000 kDa; 25 kDa to 2500 kDa; and 250 kDa to 320 kDa.

8. The method of claim 1, wherein the tdsRNA comprises
rIₙ·r(C₁₁₋₁₄U)ₙ; and rugged dsRNA (formula B).

9. The method of claim 1, wherein the composition comprises at least one pharmaceutically acceptable carrier.

10. The method of claim 1, wherein the administration is performed after primary COVID-19 infection has been resolved.

11. The method of claim 1,
wherein administering is performed after the subject was infected with SARS-COV-2 initially for a time before the administering step, wherein said time is selected from the group consisting of 30 days, 50 days, 2 months, 3 months, 4 months, 5 months, 6 months, 1 year, 2 years, and more than 2 years, and
wherein the infection was resolved before the administering step.

12. The method of claim 1, wherein administering is at least one administering method selected from the group consisting of: nasal administration; systemic administration; and intravenous administration.

13. The method of claim 1, wherein administering is performed by a delivery system comprising the tdsRNA.

14. The method of claim 13, wherein the delivery system or medical device is at least one selected from the group consisting of: a nebulizer; a sprayer; a nasal pump; a squeeze bottle; a nasal spray; a syringe sprayer or plunger sprayer; a swab; a pipette; a nasal irrigation device; and a nasal rinse.

15. The method of claim 1, or any of the preceding claims, wherein the tdsRNA is administered at a dosage of about 25 mg to 700 mg of tdsRNA per day; 20 mg to 200 mg of tdsRNA per day; 50 mg to 150 mg of tdsRNA per day; or 80 mg to 140 mg of tdsRNA per day.

16. The method of claim 1, wherein the tdsRNA is administered at a frequency selected from the group consisting of: one dose per day, one dose every 2 days, one dose every 3 days, one dose every 4 days, one dose every 5 days, one dose a week, two doses a week, three doses a week, one dose every two weeks, one dose every 3 weeks, one dose every 4 weeks, and one dose a month.

17. The method of claim 1 wherein the tdsRNA is administered two times a week and at 400 mg per administration.

18. The method of claim 1 wherein the tdsRNA is administered
two times a week at 200 mg per administration for the first 2 weeks,
two times a week at 400 mg per administration after the first 2 weeks.

19. The method of claim 1, wherein the method reduces at least one, at least two, at least three, or at least four symptoms selected from the group consisting of: difficulty concentrating or focusing; inability to exercise or be active; mental fatigue; and post exertional malaise (tiredness the day after exercise).

20. (canceled)

21. (canceled)

22. (canceled)

23. (canceled)

24. (canceled)
